(19) United States
(12) Patent Application Publication
(10) Pub. No.: US 2026/0234270 A1
(43) Pub. Date: Aug. 13, 2026

(54) METHODS FOR TREATING CONDITIONS USING ANTI-NMDA RECEPTOR ANTIBODIES

(71) Applicant: Lankenau Institute for Medical Research, Wynnewood, PA (US)

(72) Inventors: Scott K. Dessain, Wynnewood, PA (US); Fetweh H. Al-Saleem, Philadelphia, PA (US)

(73) Assignee: Lankenau Institute for Medical Research, Wynnewood, PA (US)

(21) Appl. No.: 19/104,606

(22) PCT Filed: Aug. 18, 2023
(86) PCT No.: PCT/US2023/072489

§ 371 Date: Feb. 18, 2025

Related U.S. Application Data
(60) Provisional application No. 63/371,895, filed on Aug. 19, 2022.

(51) Int. Cl. C07K 16/28; A61K 9/00; A61K 39/00; A61P 27/02; A61P 27/16; C07K 16/22

(52) U.S. Cl. CPC C07K 16/286; A61K 9/0046; A61K 9/0048; A61P 27/02; A61P 27/16; C07K 16/22; A61K 2039/505; A61K 2039/54; C07K 2317/56

(57) ABSTRACT

A method for treating retinopathy or tinnitus in a subject in need thereof, the method comprising administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

### CROSS-REFERENCE TO RELATED APPLICATIONS

This application is a National Stage Entry under 35 U.S.C. 371 of International Patent Application No. PCT/US2023/072489, filed Aug. 18, 2023, which claims the benefit under 35 USC 119 (e) of U.S. Provisional Patent Application No. 63/371,895, filed Aug. 19, 2022, which is incorporated herein by reference.

### STATEMENT REGARDING FEDERALLY SPONSORED RESEARCH OR DEVELOPMENT

This invention was made with government support under grant number R21 NS088148 awarded by the National Institutes of Health. The government has certain rights in the invention.

### REFERENCE TO AN ELECTRONIC SEQUENCE LISTING

The contents of the electronic sequence listing (MLH-134US_Seq-Listing.xml; Size: 18.0 kB; and Date of Creation: Feb. 27, 2026) are herein incorporated by reference in its entirety.

### BACKGROUND OF THE INVENTION

Tinnitus is a high pitched “ringing” in the ears that affects approximately 15% of the population and varies from mildly intrusive to highly disruptive and psychologically damaging. Chronic tinnitus has no broadly effective treatment. Down-modulation of NMDAR expression is under clinical investigation for treatment of tinnitus. Tinnitus induced by salicylate or loud noise in rats is associated with increased NMDAR activity [Guitton M J, Dudai Y. Blockade of cochlear NMDA receptors prevents long-term tinnitus during a brief consolidation window after acoustic trauma. Neural Plast. 2007; 2007:80904. doi: 10.1155/2007/80904. PubMed PMID: 18301716. PMC2246076. Guitton M J, Caston J, Ruel J, Johnson R M, Pujol R, Puel J L. Salicylate induces tinnitus through activation of cochlear nmda receptors. J Neurosci. 2003; 23 (9): 3944-52. PubMed PMID: 12736364. PMC6742173]. Esketamine, a form of the anti-NMDAR inhibitor ketamine, has shown activity in treatment of tinnitus when after intratympanic injection [Muehlmeier G, Biesinger E, Maier H. Safety of intratympanic injection of am-101 in patients with acute inner ear tinnitus. Audiol Neurootol. 2011; 16 (6): 388-97. doi: 10.1159/000322641. PubMed PMID: 21252501], and an otic gel formulation is being tested in a clinical trial.

Retinopathy is one of the most common causes of vision loss in the world and age-related macular degeneration is the most common cause of blindness in people over 50 in the U.S. While therapies have improved for some eye diseases, there is still a need for advances in methods and compositions for inhibiting or treating eye diseases, including those characterized by aberrant vascularization. Present technology to treat retinopathy acts by blocking the action of the vascular endothelial growth factor VEGF. However, certain patients have adverse reactions to the numerous unpleasant intraocular injections of anti-VEGF therapy or do not achieve satisfactory therapeutic benefit from these single agent therapies.

NMDAR signaling is involved in retinal degeneration caused by diabetes and glaucoma [Smith S B. Diabetic retinopathy and the nmda receptor. Drug News Perspect. 2002; 15 (4): 226-32. doi: 10.1358/dnp.2002.15.4.840055. PubMed PMID: 12677206. PMC3773688. Cheung W, Guo L, Cordeiro M F. Neuroprotection in glaucoma: Drug-based approaches. Optom Vis Sci. 2008; 85 (6): 406-16. doi: 10.1097/OPX.0b013e31817841e5. PubMed PMID: 18521010. PMC2597725], as well as macular degeneration [Samra et al, Implication of N-Methyl-d-Aspartate Receptor in Homocysteine-Induced Age-Related Macular Degeneration, Int J Mol Sci. 2021 August; 22 (17): 9356. doi: 10.3390/ijms22179356]. Many studies have shown that the neurotransmitter, NMDA, is itself toxic to retinal cells in vitro and in vivo. For example, NMDA is toxic to chick retinal cells in a mechanism that depends on calcium influx through the NMDAR [Ferreira I L, Duarte C B, Carvalho A P. Ca2+ influx through glutamate receptor-associated channels in retina cells correlates with neuronal cell death. Eur J Pharmacol. 1996; 302 (1-3): 15362. doi: 10.1016/0014-2999 (96) 00044-1. PubMed PMID: 8791003], and NMDA injection into the vitreous is actually one of the most commonly used models of retinal cell degeneration [Niwa M, Aoki H, Hirata A, Tomita H, Green P G, Hara A. Retinal cell degeneration in animal models. Int J Mol Sci. 2016; 17 (1). doi: 10.3390/ijms17010110. PubMed PMID: 26784179. PMC4730351]. In glaucoma, elevated homocysteine levels exert their toxic effects on retinal cells through activation of calcium signaling via the NMDAR [Ganapathy P S, White R E, Ha Y, Bozard B R, McNeil P L, Caldwell R W, et al. The role of n-methyl-d-aspartate receptor activation in homocysteine-induced death of retinal ganglion cells. Invest Ophthalmol Vis Sci. 2011; 52 (8): 5515-24. doi: 10.1167/iovs.10-6870. PubMed PMID: 21436276. PMC3176067.]. This toxicity is inhibited by the NMDAR blocker, MK-801. Similarly, the partial NMDAR blocker Memantine reduced retinal damage in a monkey model of experimental glaucoma [Gabelt B T, Rasmussen C A, Tektas O Y, Kim C B, Peterson J C, Nork T M, et al. Structure/function studies and the effects of memantine in monkeys with experimental glaucoma. Invest Ophthalmol Vis Sci. 2012; 53 (4): 2368-76. doi: 10.1167/iovs.11-8475. PubMed PMID: 22427549. PMC3833458.]. In cadaveric eyes from patients with diabetic retinopathy, elevated retinal expression of the NMDAR subunits GluN1 and GluN2 was observed [Santiago A R, Hughes J M, Kamphuis W, Schlingemann R O, Ambrosio A F. Diabetes changes ionotropic glutamate receptor subunit expression level in the human retina. Brain Res. 2008; 1198:153-9. doi: 10.1016/j.brainres.2007.12.030. PubMed PMID: 18258217.]. Furthermore, memantine has been shown to reduce retinal toxicity in the streptozotocin-induced diabetic mouse model [Kusari J, Zhou S, Padillo E, Clarke K G, Gil D W. Effect of memantine on neuroretinal function and retinal vascular changes of streptozotocin-induced diabetic rats. Invest Ophthalmol Vis Sci. 2007; 48 (11): 5152-9. doi: 10.1167/iovs.07-0427. PubMed PMID: 17962468.]

What is needed are treatments for NMDAR-related conditions, including tinnitus and retinopathy.

### SUMMARY OF THE INVENTION

Provided herein is a method of treating retinopathy in a subject in need thereof. The method includes administering an effective amount of a pharmaceutical composition comprising a recombinant, synthetic or monoclonal human antibody or an epitope binding fragment thereof that specifically binds to an N-methyl-D-aspartate Receptor (NMDAR) epitope. In certain embodiments, the antibody or fragment comprising at least one of:
(a) a heavy chain sequence encoded by a nucleic acid sequence that is at least 85% identical to SEQ ID NOs. 2, 6, or 10; or
(b) a light chain sequence encoded by a nucleic acid sequence that is at least 85% identical to SEQ ID NOs: 4, 8, or 12; or
(c) a heavy chain sequence having an amino acid sequence that is at least 85% identical to SEQ ID NOs. 1, 5, or 9; or
(d) a light chain sequence having an amino acid sequence that is at least 85% identical to SEQ ID NOs: 3, 7, or 11,
wherein the pharmaceutical composition is effective to suppress or reduce NMDA receptor mediated aberrant activity of the auditory nerve in the subject. In certain embodiments, the pharmaceutical composition comprises a 5F5, 1D1, or 2G6 antibody. In certain embodiments, the pharmaceutical composition comprises multiple antibodies.

In another aspect, a method for treating retinal bleeding in a subject in need thereof is provided. The method includes administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

Provided herein is a method of treating tinnitus in a subject in need thereof. The method includes administering an effective amount of a pharmaceutical composition comprising a recombinant, synthetic or monoclonal human antibody or an epitope binding fragment thereof that specifically binds to an N-methyl-D-aspartate Receptor (NMDAR) epitope. In certain embodiments, the antibody or fragment comprising at least one of:
(a) a heavy chain sequence encoded by a nucleic acid sequence that is at least 85% identical to SEQ ID NOs. 2, 6, or 10; or
(b) a light chain sequence encoded by a nucleic acid sequence that is at least 85% identical to SEQ ID NOs: 4, 8, or 12; or
(c) a heavy chain sequence having an amino acid sequence that is at least 85% identical to SEQ ID NOs. 1, 5, or 9; or
(d) a light chain sequence having an amino acid sequence that is at least 85% identical to SEQ ID NOs: 3, 7, or 11,
wherein the pharmaceutical composition is effective to suppress or reduce NMDA receptor mediated aberrant activity of the auditory nerve in the subject. In certain embodiments, the pharmaceutical composition comprises a 5F5, 1D1, or 2G6 antibody. In certain embodiments, the pharmaceutical composition comprises multiple antibodies.

Still other aspects and advantages of these compositions and methods for making the compositions and using the compositions are described further in the following detailed description of the preferred embodiments thereof.

### BRIEF DESCRIPTION OF THE DRAWINGS

FIG. 1 shows antibody binding in mouse retina under normoxic conditions. Antibodies 5F5, 2G6, and 1D1 bind to an NMDAR epitope, while 9H2 is specific for poliovirus and serves as a control. None of the anti-NMDAR antibodies or the control antibody bound to mouse retina under normoxic conditions. Left panels show DAPI staining, middle panels show antibody staining, right panels show merged imaging.

FIG. 2 shows antibody binding in mouse retina under hypoxic conditions. The 5F5, 2G6, and 1D1 antibodies show binding in outer segments of mouse retina under hyperoxic conditions. Control antibody (9H2) shows no binding. Left panels show DAPI staining, middle panels show antibody staining, right antibodies show merged imaging.

FIGS. 3A and 3B show whole mouse eyes treated with PBS (3A) or an antibody against NMDAR (3B) as described in Example 7.

FIG. 4 shows antibody binding in mouse retina in a model of retinitis pigmentosa. Left panels show DAPI staining, middle panels show antibody staining, right antibodies show merged imaging.

### DETAILED DESCRIPTION

Methods of treating conditions using anti-N-methyl-D-aspartate Receptor (NMDAR) antibodies are provided herein. Described herein are new uses for monoclonal human antibodies specific for the ionotropic NMDA Receptor (NMDAR), specifically NMDARs that incorporate the NR1 (GluN1) subunit in their structure. The monoclonal antibodies inhibit NMDAR activity and can thus be used as pharmaceuticals to alleviate conditions that involve pathologic NMDAR activation. The three antibodies described are structurally distinct and bind non-overlapping sites on the NMDAR, such that they can be administered singly or in 2- or 3-antibody combinations. Specifically referred to herein is inhibition of the ionotropic NMDAR, as encoded by the genes GRIN1, GRIN2, and/or GRIN3. In contrast to small molecules that inhibit NMDAR, monoclonal antibodies have long half-lives and do not efficiently cross the blood-brain barrier to interact with CNS NMDARs. Useful antibodies are described in WO 2020/204977, which is incorporated herein by reference in its entirety. In certain embodiments, the condition is tinnitus. In other embodiments, the condition is retinopathy.

In one aspect, provided herein is a treatment for an otic disorder, e.g., tinnitus. Tinnitus is a high pitched “ringing” in the ears that affects approximately 15% of the population and varies from mildly intrusive to highly disruptive and psychologically damaging [3]. The causes of tinnitus are likely to be multifactorial, but important evidence shows that in some cases over activation of the ionotropic NMDAR is involved. Provided herein is the use of an anti-NDMAR antibody for treatment of tinnitus.

In another aspect, provided herein is a treatment for retinopathy, including retinopathy caused by glaucoma, diabetes and macular degeneration. In certain embodiments, the treatment inhibits, reduces, or prevents retinal neovascularization.

### Components Utilized in the Compositions and Methods

In the descriptions of the compositions and methods discussed herein, the various components can be defined by use of technical and scientific terms having the same meaning as commonly understood by one of ordinary skill in the art to which this invention belongs and by reference to published texts. Such texts provide one skilled in the art with a general guide to many of the terms used in the present application. The definitions contained in this specification are provided for clarity in describing the components and compositions herein and are not intended to limit the claimed invention.

### A. Subject

“Patient” or “subject” or “individual” as used herein means a mammalian animal, including a human, a veterinary or farm animal, a domestic animal or pet, and animals normally used for clinical research. In one embodiment, the subject of these methods and compositions is a human. In one embodiment, the subject has an ocular disease. In another embodiment, the subjects have an ocular disease and have yet to be treated with any therapy. In another embodiment, the subject has an ocular disease and is treated with conventional methodologies, e.g., administration of VEGF inhibitors intraocularly, but is not responding to the treatment optimally or in a manner sufficient to achieve a sufficient therapeutic benefit. In another embodiment, the subject having said ocular disease is receiving administration of VEGF inhibitors or blockers but is not achieving the desired therapeutically maximal response that been observed in other patients upon the administration of a VEGF blocker or inhibitor monotherapy.

In another embodiment, the subject has an otic disease. In another embodiment, the subjects have an otic disease and have yet to be treated with any therapy. In another embodiment, the subject has an otic disease and is treated with conventional methodologies, e.g., behavioral therapy, but is not responding to the treatment optimally or in a manner sufficient to achieve a sufficient therapeutic benefit. In another embodiment, the subject having said otic disease is receiving standard therapy but is not achieving the desired therapeutically maximal response that been observed in other patients.

### B. Ocular Disease

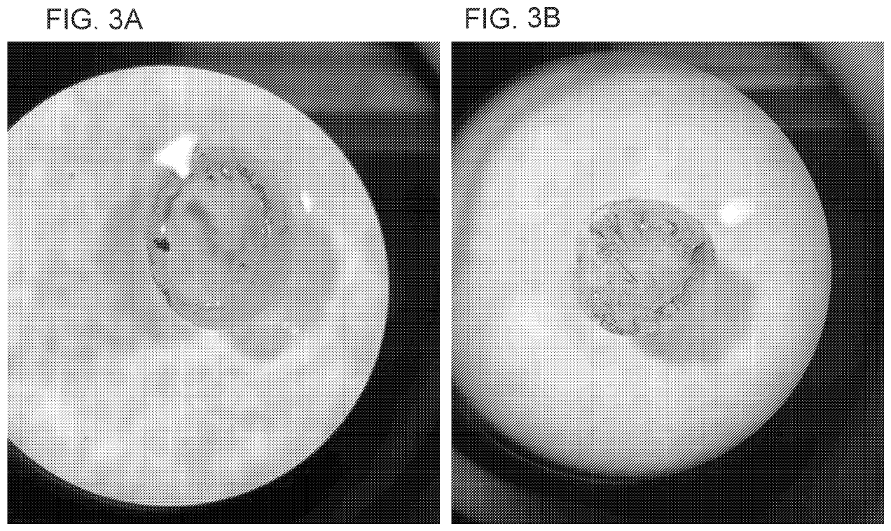

NMDAR is not expressed in ocular cells under normal conditions. As shown for the first time herein, NMDAR is however activated under hypoxic conditions in the mouse model of oxygen-induced retinopathy. See, e.g., Kim C B, D'Amore P A, Connor K M. Revisiting the mouse model of oxygen-induced retinopathy. Eye Brain. 2016; 8:67-79. Epub 2016 May 20, which is incorporated herein by reference. The oxygen induced retinopathy (OIR) model is the gold standard preclinical model for research in ocular vascular pathologies and is one of the most widely cited disease models in ophthalmology and vascular biology research. The antibodies described herein are known to cause down-modulation of NMDAR expression on the surface of cells and are shown herein to prevent abnormal vascularization and hemorrhage (FIGS. 3A and 3B) in the OIR.

There is support in the scientific literature for the involvement of NMDAR signaling in retinal degeneration caused by diabetes and glaucoma [6,7]. Many studies have shown that the neurotransmitter, NMDA, is itself toxic to retinal cells in vitro and in vivo. For example, NMDA is toxic to chick retinal cells in a mechanism that depends on calcium influx through the NMDAR [8], and NMDA injection into the vitreous is actually one of the most commonly used models of retinal cell degeneration [9].

In glaucoma, elevated homocysteine levels exert their toxic effects on retinal cells through activation of calcium signaling via the NMDAR [10]. This toxicity is inhibited by the NMDAR blocker, MK-801. Similarly, the partial NMDAR blocker Memantine reduced retinal damage in a monkey model of experimental glaucoma [11]. In cadaveric eyes from patients with diabetic retinopathy, elevated retinal expression of the NMDAR subunits GluN1 and GluN2 was observed [12]. Note, GluN1 is the protein bound by the ANRE antibodies of the present invention. Furthermore, memantine has been shown to reduce retinal toxicity in the streptozotocin-induced diabetic mouse model [13].

By the term “ocular disease” is meant a retinopathy. Examples of ocular diseases include, without limitation, glaucoma, and retinopathy (e.g., diabetic retinopathy, vascular retinopathy glaucoma, macular degeneration, retinopathy of prematurity, hypertensive retinopathy, and central serous retinopathy). In one embodiment, an ocular disease is characterized by neovascularization, i.e., new or abnormal blood vessel formation in a tissue or part of the eye, or excessive blood vessel formation is a tissue or part of the eye. In one embodiment, the ocular disorder is a retinopathy. In a particular embodiment, the ocular disease is characterized by abnormal/aberrant vascularization. In a particular embodiment, the ocular disease is characterized by leaky vessels. In a particular embodiment the ocular disease is characterized by intraocular neovascularization. The intraocular neovascularization may be, without limitation, neovascularization of the optic disc, iris, retina, choroid, cornea, and/or vitreous humour. Examples of ocular diseases include, without limitation, glaucoma, pannus, pterygium, macular edema, macular degeneration (e.g., age-related macular degeneration), retinopathy (e.g., diabetic retinopathy, vascular retinopathy, retinopathy of prematurity), diabetic retinal ischemia, diabetic macular edema, retinal degeneration, retrolental fibroplasias, retinoblastoma, corneal graft neovascularization, central retinal vein occlusion, pathological myopia, ocular tumors, uveitis, inflammatory diseases of the eye, and proliferative vitreoretinopathy. In a particular embodiment, the ocular disease is selected from the group consisting of retinopathy (e.g., retinopathy of prematurity, diabetic retinopathy (e.g., proliferative diabetic retinopathy)) and macular degeneration (e.g., dry or wet macular degeneration). In certain embodiments, the ocular disease is retinitis pigmentosa.

### C. Otic Disease

By the term “otic disease or disorder” is meant a disease that affects the ear or hearing of the subject. Otic disorders are numerous and include ototoxicity, excitotoxicity, sensorineural hearing loss, noise induced hearing loss, Meniere's Disease/Syndrome, endolymphatic hydrops, labyrinthitis, Ramsay Hunt's Syndrome, vestibular neuronitis, tinnitus and microvascular compression syndrome. In particular embodiments, the otic disorder is tinnitus.

### D. Anti-NMDAR Antibody

The methods described herein utilize an anti-NMDAR antibody. In one embodiment, an “antibody” refers to an intact immunoglobulin, such as an IgG, or to an antigen binding portion thereof that competes with the intact antibody for specific binding, unless otherwise specified. In one embodiment, the antibody is an IgG1, IgG2, IgG3 or IgG4. An antibody (e.g., an antibody, an antibody heavy chain, an antibody light chain, or any fragment or modification thereof) comprises three Complementarity-Determining Regions (CDRs, also known as HV, hypervariable regions, namely CDR1, CDR2, CDR3, from N-terminal to C-terminal, or 5′ to 3′ when corresponding nucleic acid sequence is referred to), and four framework regions (FRs, namely FR1, FR2, FR3 and FR4, from N-terminal to C-terminal, or 5′ to 3′ when corresponding nucleic acid sequence is referred to). See, e.g., Janeway, Charles A Jr; Travers, Paul; Walport, Mark; Shlomchik, Mark J (2001). Immunobiology: The Immune System in Health and Disease (5 ed.). New York: Garland Science. ISBN 0-8153-3642-X, which is incorporated herein by its entirety. It would be understood that in the antibody construct, CDRs are arranged non-consecutively, not immediately adjacent to each other, and may be separated by an FR. As part of the variable chain in an antibody construct and T cell receptors generated by B-cells and T-cells respectively, CDRs are where an antigen specifically binds.

In certain embodiments, the antibody or fragment includes a monoclonal antibody, such as those described in WO 2020/204977. In one embodiment, the antibody is the 5F5 antibody. In another embodiment, the antibody is the 2G6 antibody. In yet another embodiment, the antibody is the 1D1 antibody. The SEQ ID NOs. of antibodies 5F5, 2G6, and 1D1 are reproduced in Table 1 below.

TABLE 1

| SEQ ID NO: | Name | Type |
|---|---|---|
| 1 | 5F5 Heavy Chain | Protein |
| 2 | 5F5 Heavy Chain | DNA |
| 3 | 5F5 Light Chain | Protein |
| 4 | 5F5 Light Chain | DNA |
| 5 | 1D1 Heavy Chain | Protein |
| 6 | 1D1 Heavy Chain | DNA |
| 7 | 1D1 Light Chain | Protein |
| 8 | 1D1 Light Chain | DNA |
| 9 | 2G6 Heavy Chain | Protein |
| 10 | 2G6 Heavy Chain | DNA |
| 11 | 2G6 Light Chain | Protein |
| 12 | 2G6 Light Chain | DNA |

Useful antibodies can also include a synthetic antibody, a recombinant antibody, a chimeric antibody, a humanized antibody, a human antibody, a CDR-grafted antibody, a multispecific binding construct that can bind two or more epitopes, a dual specific antibody, a bi-specific antibody, a multi-specific antibody, an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′) 2 construct, an Fc construct, a monovalent or bivalent construct from which domains non-essential to monoclonal antibody function have been removed, a single-chain molecule containing one VL (variable region of light chain), one VH (variable region of heavy chain) antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, or any recombinant versions thereof. Definitions and examples of these types of structures are found in the art and in, e.g., U.S. Pat. No. 9,902,772, incorporated by reference herein.

The term “recombinant antibody” refers to an antibody that is generated by cloning the immune-specific heavy and light antibody coding sequences into a vector. In one embodiment, the vector is designed for high-yield mammalian expression. The resulting vectors are introduced into expression hosts (e.g., bacteria, virus, yeast or mammalian) for the manufacturing of high-quality functional antibodies. Generally, the coding sequence is not naturally associated with the host cell. Recombinant antibodies have glycosylation patterns that differ from the glycosylation pattern of an antibody having the same sequence if it were to exist in nature. In one embodiment, a recombinant antibody is expressed in a mammalian host cell which is not a human host cell. Notably, individual mammalian host cells have unique glycosylation patterns. Recombinant antibodies can be constructed in vitro by forming an Ig-framework through cloning of scFV or Fab or can be produced from an existing hybridoma. In hybridoma-based recombinant antibody generation, mouse, rat, and rabbit models are commonly used. However, as long as the appropriate oligonucleotide primers are available, recombinant antibodies can be developed from any species.

Recombinant antibodies can also be produced within the body of a subject who has received a nucleic acid that directs expression of the antibody in the subject's tissues. The nucleic acid may be DNA, mRNA, or related molecule, introduced into the subject's body through direct injection, electrical stimulation, or within a viral-derived vector, such as an AAV, VSV-G or lentivirus, in such a way that the nucleic acid directs production of the within the subject's body. See, Nanegrungsunk et al, New frontiers of retinal therapeutic intervention: a critical analysis of novel approaches. Ann Med. 2022 December; 54 (1): 1067-1080. doi: 10.1080/07853890.2022.2066169 and Liu and Yang, Inner Ear Drug Delivery for Sensorineural Hearing Loss: Current Challenges and Opportunities. Front Neurosci. 2022 May 24; 16:867453. doi: 10.3389/fnins.2022.867453, which are incorporated herein by reference.

As used herein, an “antibody mimic” or an “antibody equivalent” refers to affibodies, i.e., a class of engineered affinity proteins, generally small (~6.5 kDa) single domain proteins that can be isolated for high affinity and specificity to any given target, aptamers, polypeptide molecules that bind to a specific target, an affilin, an affitin, an affimer, an alphabody, an anticalin, an avimer, a DARPin (designed ankyrin repeat proteins), a Fynomer, a Kunitz domain peptide, a monobody, a peptabody and others known in the art.

In certain embodiments, the anti-NMDAR antibody includes the monoclonal IgG immunoglobulins 5G5, 2G6 and 1D1, comprising two full-length heavy chains (each chain comprising a variable region and a constant region) and two full-length light chains (each chain comprising a variable region and a constant region), as well as modifications, antigen/epitope binding fragments, as well as “antibody mimics” or “antibody equivalents” or constructs of fragments encoded by one or more of SEQ ID Nos: 1-12. In one embodiment, the antibody or epitope binding fragments as described herein refers to an anti-NMDAR antibody or fragment encoded by a nucleic acid sequence at least 85% identical to one of SEQ ID NO: 2, 4, 6, 8, 10, or 12.

In another embodiment, the anti-NMDAR antibody includes the monoclonal IgG immunoglobulins 5G5, 2G6 and 1D1, comprising two full-length heavy chains (each chain comprising a variable region and a constant region) and two full-length light chains (each chain comprising a variable region and a constant region), as well as modifications, antigen/epitope binding fragments, as well as antibody mimics” or “antibody equivalents” or constructs of fragments comprising at least one amino acid sequence of SEQ ID NO: 1, 3, 5, 7, 9, or 11.

In one embodiment, the antibody or epitope binding fragments as described herein refers to an anti-NMDAR antibody or fragment having a heavy chain amino acid sequences at least 85% identical to one of SEQ ID NO: 1, 5 or 9. In another embodiment, the antibody or epitope binding fragments as described herein refers to an anti-NMDAR antibody or fragment having a light chain amino acid sequence at least 85% identical to one of SEQ ID NO: 3, 7, or 11. By “at least 85% identical” encompasses at least 86%, 87%, 88%, 89%, 90%, 91%, 92%, 93%, 94%, 95%, 96%, 97%, 98%, 99%, up to 100% sequence identity with the identified reference SEQ ID NO.

As used herein, a “modification” of an antibody refers to an antibody heavy chain or light chain amino acid sequence, in which wherein one or more amino acid residues are inserted into, deleted from, and/or substituted into the reference amino acid sequence, e.g., any of amino acid sequence encoding the variable light or heavy chains, and/or CDRs of antibodies 5F5, 2G6 or 1D1. See Table 2 and WO 2020/204977. One such modification is the replacement of one amino acid in such a sequence, e.g., any of amino acid sequences encoded by SEQ ID NO: 2, 4, 6, 8, 10, or 12, or amino acid sequences of 1, 3, 5, 7, 9, or 11, with a conservative amino acid. Other modifications include, for example, fusion proteins formed by fusing the heavy chain of a selected antibody into an Ig backbone. Still another modification includes an anti-NMDAR antibody that has been modified via conjugation to another chemical moiety (such as, for example, polyethylene glycol or albumin, e.g., human serum albumin), phosphorylation, and glycosylation. In another embodiment, a modification of any of antibodies 5F5, 2G6 or 1D1 is a single chain human antibody, having a variable domain region from a heavy chain and a variable domain region from a light chain and a peptide linker connecting the heavy chain and light chain variable domain regions.

Methods for producing such antibodies and antibody fragments are well-known in the art. Indeed, commercial vectors for certain antibody and antibody fragment constructs are available. The antibody may also be a protein (e.g., a fusion protein) comprising at least one antibody or antibody fragment. In a particular embodiment, the antibody comprises an Fc region.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody.

The term “NMDAR epitope” as used herein refers to the portion of an NMDAR protein or any naturally occurring or synthetic or recombinant amino acid sequence that is capable of specifically complexing with one or more of the antibodies 5F5, 2G6 or 1D1, or epitope binding fragments or modified antibodies encoded by one or more of SEQ ID NOs: 2, 4, 6, 8, 10, or 12, or by sequences at least 85% identical to SEQ ID NOs: 2, 4, 6, 8, 10, or 12, or having an amino acid sequence of SEQ ID Nos: 1, 3, 5, 7, 9, or 11 or having a sequence at least 85% identical to one of more of SEQ ID Nos: 1, 3, 5, 7, 9, or 11, as described herein.

As used herein, the term “immunologically specific” refers to antibodies, that bind to one or more epitopes of a protein or compound of interest, but which do not substantially recognize and bind other molecules in a sample containing a mixed population of antigenic biological molecules.

The term “isolated” designates a biological material (nucleic acid or protein) that has been removed from its original environment (the environment in which it is naturally present). For example, a polynucleotide present in its natural state in a plant or an animal is not isolated, however the same polynucleotide separated from the adjacent nucleic acids in which it is naturally present, is considered “isolated”. The term “purified” does not require the material to be present in a form exhibiting absolute purity, exclusive of the presence of other compounds.

The terms “percent (%) identity”, “sequence identity”, “percent sequence identity”, or “percent identical” in the context of amino acid sequences or nucleotide sequences refers to the residues in the two sequences which are the same when aligned for correspondence. Percent identity may be readily determined for amino acid sequences or nucleotide sequences over the full-length of a protein, polypeptide, or encoding region thereof, e.g., about 15 amino acids, about 150 amino acids, or a peptide fragment thereof or the corresponding nucleic acid sequence coding sequences. A suitable amino acid fragment may be at least about 4 amino acids in length and may be up to about 200 or up to about 700 amino acids or nucleotide fragments of from about 12 nucleotides to about 600 to 2100 nucleotides. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., (THOMPSON et al. 1999).

### Pharmaceutical Compositions

Provided herein are methods that utilize pharmaceutical compositions that include anti-N-methyl-D-aspartate Receptor (NMDAR) antibodies useful in the treatment of certain conditions as described herein. In certain embodiments, the NMDAR antibodies are those cloned from an ANRE patient. Such antibodies are known in the art, or may be discovered. Three monoclonal Abs are identified as 5F5, 2G6 and 1D1, in WO 2020/204977 which is incorporated herein by reference in its entirety. The antibodies can be administered individually or in combinations of two or three, simultaneously or sequentially. The antibodies may be converted into bispecific antibodies, in which NMDAR-binding domains of two of the mAbs are combined in a single molecule. Labrijn et al, Bispecific antibodies: a mechanistic review of the pipeline, Nat Rev Drug Discov, 2019 August; 18 (8): 585-608. doi: 10.1038/s41573-019-0028-1. The nucleic acid sequences encoding the variable heavy and light chains of antibodies 5F5, 2G6 and 1D1 are provided in the Sequence Listing. In certain embodiments, “variable heavy” or “light chain” refers to variable region of a heavy or light immunoglobulin chain.

The corresponding amino acid sequences of the variable heavy and light chains of antibodies 5F5, 2G6 and 1D1 are also provided in the Sequence Listing. Table 2 reproduces the amino acid sequences corresponding to the variable regions of the heavy and light chains, with their respective complementarity-determining regions (CDR1, CDR2, and CDR3) underlined.

TABLE 2

| mAb 5F5 Amino Acid Sequences | | |
|---|---|---|
| Variable | QVQLVESGGGVVRPGGSLRLSCAASGFTFST | SEQ ID |
| Heavy | YSLHWVRQAPGKGLEWVGVILYDGSKKYYAD | NO: 1 |
| Chain | SVRGRFTISRDNSKSTLNLDMSSLRPDDTAV | |
| | YYCARDPIAVAPRPSGMVPQGFDYWGQGTLV | |
| | TVSS | |
| Variable | QSVVTQPPSASGTPGQRVTISCSGSSSNIGR | SEQ ID |
| Light | NFVFWYRQLPGTAPKVLIYKNIQRPSGVPDR | NO: 3 |
| Chain | ISGSRSGSSASLAISGLRSEDEADYYCASWD | |
| | DSLSGVVFGGGTKLTVL | |

mAb 2G6 Amino Acid Sequences

| | | |
|---|---|---|
| Variable | QVQLQESGGGVVQPGRSLRLSCAASGFSFNA | SEQ ID |
| Heavy | FAMHWVRQAPGKGLEWVARISHYGSDDYYAD | NO: 9 |
| Chain | SVKGRFTISRDNSQNTLFLQMNSLKAEDTGV | |
| | YYCWRGFTLVRGVISRNPINRFSGMDVWGQG | |
| | TTVTVS | |
| Variable | SYELMQPPSVSVSPGQTARITCSGDALPKKY | SEQ ID |
| Light | AYWYQQKSGQAPILVIYEDNKRPSGIPERFS | NO: 11 |
| Chain | GSSSGTMATLTISGAQVEDEADYFCYSTDSS | |
| | GNHGVFGGGTKLTVL | |

mAb 1D1 Amino Acid Sequences

| | | |
|---|---|---|
| Variable | QVQLVESGGGVVQSGRSLRLSCAASGFAFHT | SEQ ID |
| Heavy | FTIHWVRQAPGKGLDWVTAIYFDGTKKYYAD | NO: 5 |
| Chain | SVKGRFTVSRDNSKNTVYLQMNGLRGEDTAV | |
| | YYCARARYSYGLSFDYWGQGTPVTVSS | |
| Variable | QSVVTQPPSVSAAPGQKVTISCSASSSYLGS | SEQ ID |
| Light | NYVSWYQQLPGTAPKLLIYDNNKRSSGISDR | NO: 7 |
| Chain | ESGSKSGTSATLDITGLQTGDEADYYCGAWD | |
| | SSLSVVVFGGGTKLTVL | |

In certain embodiments, a CDR of any one of SEQ ID NOs: 10-12 is the one shown in Table 2 truncated with 1, 2, or 3 amino acids in the N terminus and/or the C terminus. In certain embodiments, the antibody, or a variant thereof, or an epitope binding fragment thereof comprises 1, 2, 3, 4, 5, or 6 CDR(s) as described. As used herein, the complementarity-determining region (CDR) refers to part of the variable chains in antibodies or T cell receptors, which binds to the corresponding epitope. Such CDR may be determined via experiments or via various predicating tools, such as www.imgt.org/IMGT_vquest/analysis. As used herein, an epitope binding fragment refers to a fragment of an antibody which is determined to be bound to an epitope. Such determination may be performed experimentally using, for example, ELISA or other methods known in the art or via various predicating tools such as IMGT.org.

In certain embodiments, the antibody binds to an epitope in a GluN1 subunit of the NMDAR. In another embodiment, the antibody binds to an epitope in a GluN2 subunit of NMDAR.

Other anti-NMDAR1 antibodies are known in the art. Such antibodies include the NMDAR1 Antibody (R1JHL) from Novus Bio, that was shown to react with human protein (Anna P. Mashkina, et al (2010). NMDA Receptors are Expressed in Lymphocytes Activated Both In Vitro and In Vivo., 30 (6), 901-907).

Utilizing any of the nucleotide sequences encoding the heavy chain variable region of 5F5, 2G6, and 1D1, the light chain variable region of 5F5, 2G6, and 1D1, their encoded amino acid sequences for the heavy chain variable region of 5F5, 2G6, and 1D1, the light chain variable region of 5F5, 2G6, and 1D1, or nucleotide or amino acid sequences sharing at least about 80% (for example, about 80%, about 85%, about 90%, about 95%, about 96%, about 97%, about 98%, or about 99%), at least about 85%, at least about 90%, at least about 95%, at least about 96%, at least about 97%, at least about 98%, at least about 99%, or at least about 99.9% sequence identity therewith, other antibodies or fragments that specifically bind to the same NMDAR epitope(s), can be generated for use in the methods described herein. In certain embodiments, the antibody or epitope binding fragment thereof as described herein comprise one or more of the CDRs as illustrated in Table 2. In certain embodiments, a described amino acid sequence sharing a certain percentage (which is less than 100%) identity to any one of SEQ ID NOs: 1, 3, 5, 7, 9, or 11 is different from the sequence with the corresponding SEQ ID NO in the region other than the CDR illustrated in Table 2.

In certain embodiments, more than one antibody or antibody fragment is employed. In certain embodiments, it is desirable to utilize multiple antibodies, wherein each binds to a non-overlapping site on NMDAR1. For example, the composition may include two or more of 5F5, 2G6 or 1D1, or fragments thereof.

### Methods of Treatment

In one aspect, a method for treating an otic disorder in a subject in need thereof is provided. Otic disorders produce symptoms which include but are not limited to hearing loss, nystagmus, vertigo, tinnitus, inflammation, infection and congestion. Otic disorders are numerous and include ototoxicity, excitotoxicity, sensorineural hearing loss, noise induced hearing loss, Meniere's Disease/Syndrome, endolymphatic hydrops, labyrinthitis, Ramsay Hunt's Syndrome, vestibular neuronitis, tinnitus and microvascular compression syndrome. In particular embodiments, the otic disorder is tinnitus. In certain embodiments, the otic disorder is drug-induced hearing loss. See, e.g., Juan Hong, et al. N-Methyl-D-Aspartate Receptors Involvement in the Gentamicin-Induced Hearing Loss and Pathological Changes of Ribbon Synapse in the Mouse Cochlear Inner Hair Cells, Neural Plasticity, vol. 2018, Article ID 3989201, 16 pages, 2018, which is incorporated herein by reference. In another embodiment, the otic disorder is noise induced hearing loss. See, e.g., Chen G D, et al. NMDA receptor blockage protects against permanent noise-induced hearing loss but not its potentiation by carbon monoxide. Hear Res. 2001 April; 154 (1-2): 108-15, which is incorporated herein by reference.

The method includes administering an effective amount of a pharmaceutical composition comprising a recombinant, synthetic, or monoclonal human antibody or an epitope binding fragment thereof that specifically binds to NMDAR. In certain embodiment, the pharmaceutical composition comprises at least one recombinant, synthetic or monoclonal human antibody or fragment thereof that binds to an N-methyl-D-aspartate Receptor (NMDAR) epitope, wherein said antibody or fragment comprises at least one heavy chain sequence or light chain sequence of 5F5, 1D1, or 2G6. Any of the antibodies described above and based on these sequences can be used in pharmaceutical compositions, as well as anti-NMDAR1 antibodies known in the art. In one embodiment, a pharmaceutical composition contains a mixture of two or more of said antibodies or epitope binding antibody fragments described herein. In still another embodiment, a pharmaceutical composition contains an additional anti-NMDAR antibody or antibody fragment that binds to a different NMDAR epitope than do the antibodies and fragments described here.

In certain embodiments, the antibodies are administered after the onset of hearing loss. In certain embodiments, the antibodies are administered after at least a 5%, 10%, 15%, 20%, 25%, 30%, 35%, 40%, 45%, 50%, 55%, 60%, 65%, 70%, 75%, 80%, 85%, 90%, or 95% reduction of hearing. In certain embodiments, the antibodies are administered after about a 5%, 10%, 15%, 20%, 25%, 30%, 35%, 40%, 45%, 50%, 55%, 60%, 65%, 70%, 75%, 80%, 85%, 90%, or 95% reduction of hearing. In other embodiments, the antibodies are administered prior to onset of measurable hearing. In other embodiments, the antibodies are administered prophylactically, e.g., prior to exposure to ototoxic drugs (chemotherapy patients or those needing some antibiotics) or noise, for example, soldiers in battle.

In another aspect, a method for treating a retinopathy in a subject in need thereof is provided. In one embodiment, the retinopathy is glaucoma. In another embodiment, the retinopathy is diabetic retinopathy. In another embodiment, the retinopathy is a proliferative or vascular retinopathy. In another embodiment, the subject has retinitis pigmentosa. The method includes administering an effective amount of a pharmaceutical composition comprising a recombinant, synthetic, or monoclonal human antibody or an epitope binding fragment thereof that specifically binds to an NMDAR epitope, as described herein. In certain embodiment, the pharmaceutical composition comprises at least one recombinant, synthetic or monoclonal human antibody or fragment thereof that binds to an N-methyl-D-aspartate Receptor (NMDAR) epitope, wherein said antibody or fragment comprises at least one heavy chain sequence or light chain sequence of 5F5, 1D1, or 2G6. Any of the antibodies described above and based on these sequences can be used in pharmaceutical compositions, as well as anti-NMDAR1 antibodies known in the art. In one embodiment, a pharmaceutical composition contains a mixture of two or more of said antibodies or epitope binding antibody fragments described herein. In still another embodiment, a pharmaceutical composition contains an additional anti-NMDAR antibody or antibody fragment that binds to a different NMDAR epitope than do the antibodies and fragments described here.

In certain embodiments, the antibodies are administered after the onset of vision loss. In certain embodiments, the antibodies are administered after at least a 5%, 10%, 15%, 20%, 25%, 30%, 35%, 40%, 45%, 50%, 55%, 60%, 65%, 70%, 75%, 80%, 85%, 90%, or 95% reduction of vision. In certain embodiments, the antibodies are administered after about a 5%, 10%, 15%, 20%, 25%, 30%, 35%, 40%, 45%, 50%, 55%, 60%, 65%, 70%, 75%, 80%, 85%, 90%, or 95% reduction of vision. In other embodiments, the antibodies are administered prior to onset of measurable vision loss. In other embodiments, the antibodies are administered prophylactically.

In certain embodiments, the antibodies are administered after the onset of photoreceptor damage or loss. In certain embodiments, the antibodies are administered prior to the onset of photoreceptor damage or loss. In certain embodiments, the antibodies are administered after the onset of pathologic vascularization. In certain embodiments, the antibodies are administered prior to the onset of pathologic vascularization.

### Administration

In certain embodiments, the pharmaceutical composition is administered to the ear for treatment of tinnitus. Suitable delivery methods include any route of administration that allows the pharmaceutical composition to contact the cochlea. In certain embodiments, the administration is systemic, such as intravenous. However, in certain embodiments, administration to the ear is preferred. In certain embodiments, the pharmaceutical composition is administered to the middle ear. In other embodiments, the pharmaceutical composition is administered to the inner ear. Intratympanic injection may be used. In intratympanic injection, medication is given through the ear drum into the inner ear. By injecting medication through the ear drum, the middle ear space is filled with fluid containing the drug of choice. The inner ear absorbs some of the medication across the round window. The round window is a naturally occurring opening in the bone of the inner ear. Only a membrane separates the inner ear and middle ear within the round window. Medications can be slowly absorbed through this membrane into the inner ear. In other embodiments, intracochlear delivery is utilized.

In other embodiments, the pharmaceutical composition can be in the form of a lipid coated iron oxide nanoparticle that comprises the anti-NMDAR1 antibody. The pharmaceutical composition comprising the iron oxide nanoparticles is administered to the ear and magnetically pushed or pulled to the treatment site, i.e., inner ear. Exemplary magnetic nanoparticles are described in WO 2021/081251 and WO 2019/006440, which are incorporated herein by reference.

In certain embodiments, the pharmaceutical composition is administered to the eye for treatment of retinopathy. In one embodiment, the method involves administration via subretinal injection to the RPE, photoreceptor cells or other ocular cells. In one embodiment, the method involves administration via subretinal injection. In another embodiment, intravitreal injection to ocular cells is employed. In still another method, injection via the palpebral vein to ocular cells may be employed. In still another embodiment, suprachoroidal injection to ocular cells may be employed.

Still other methods of administration may be selected by one of skill in the art given this disclosure. By “administering” or “route of administration” is delivery of a therapy described herein (e.g., a composition comprising an anti-NMDAR1 antibody), with or without a pharmaceutical carrier or excipient, of the subject. Routes of administration may be combined, if desired. In some embodiments, the administration is repeated periodically. Direct delivery to the eye (optionally via ocular delivery, subretinal injection, intra-retinal injection, intravitreal, topical), ear (optionally via intratympanic delivery) or delivery via systemic routes, e.g., intraarterial, intraocular, intravenous, intramuscular, subcutaneous, intradermal, and other parental routes of administration are contemplated.

As used herein, the term “ocular cells” refers to any cell in, or associated with the function of, the eye. The term may refer to any one of photoreceptor cells, including rod, cone and photosensitive ganglion cells or retinal pigment epithelium (RPE) cells. In one embodiment, the ocular cells are the photoreceptor cells. In another embodiment, the ocular cells are the RPE.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline).

In certain embodiments, the composition comprises a dispersing agent or viscosity modulating agent. “Dispersing agents,” and/or “viscosity modulating agents” are materials that control the diffusion and homogeneity of the antibody through liquid media. Examples of diffusion facilitators/dispersing agents include but are not limited to hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol has a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also be used as dispersing agents. Dispersing agents useful in liposomal dispersions and self-emulsifying dispersions of the antibodies disclosed herein are dimyristoyl phosphatidyl choline, natural phosphatidyl choline from eggs, natural phosphatidyl glycerol from eggs, cholesterol and isopropyl myristate.

Dosage of the composition will depend, among other things, on the condition of the subject, the particular indication being treated, the route of administration, the formulation of the therapeutic agent employed, etc. For example, the dosage will typically be about 1 to 10 micrograms per kilogram subject body weight. The specific dosage of the antibody is not critical, as long as it is effective to result in some beneficial effects in some individuals within an affected population. In general, the dosage may be as low as about 1, 5, 10, 20 or 50 micrograms per kilogram subject body weight, or lower, and as high as about 200, 500, 1000, 2000 or 5000 micrograms per kilogram subject body weight, or even higher. The terms “effective amount” or “therapeutically effective amount,” as used herein, refer to a sufficient amount of the antibody being administered that would be expected to relieve to some extent one or more of the symptoms of the disease or condition being treated. For example, the result of administration of the antibody disclosed herein is reduction and/or alleviation of the signs, symptoms, or causes of tinnitus or balance disorders. For example, in another embodiment, the result of administration of the antibody disclosed herein is reduction and/or alleviation of the signs, symptoms, or causes of retinopathy. For example, an “effective amount” for therapeutic uses is the amount of antibody, including a formulation as disclosed herein required to provide a decrease or amelioration in disease symptoms without undue adverse side effects. The term “therapeutically effective amount” includes, for example, a prophylactically effective amount. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician.

The compositions described herein may be administered on a regimen as determined by a physician. In certain embodiments, the compositions are administered more than once per day. In other embodiments, the compositions are administered daily. In other embodiments, the compositions are administered 2, 3, 4, 5, 6, or more times per week. In other embodiments, the compositions are administered 1, 2, 3, 4, 5 or more times per month. In other embodiments, the compositions are administered every 2, 3, 4, 5, 6, 7, 8, 9, or 10 weeks more times per month. In other embodiments, the compositions are administered 1, 2, 3, 4, 5, 6, 7, 8, 9, 10, 11, 12 or more times per year.

The compositions described herein may optionally be administered in conjunction with other, different, therapeutic agents useful in the treatment of the disorders or conditions described herein. For example, in the case of retinopathy, the composition may be administered with a VEGF inhibitor, e.g., an anti-VEGF antibody. In other embodiments, the other agent is a steroid.

In other embodiments, the composition is administered with a treatment for an otic disorder, e.g., tinnitus. Such treatments include lidocaine, selective neurotransmitter reuptake inhibitors, such as nortriptyline, sertraline, and paroxetine, benzodiazepines, and ketamines, such as esketamine.

As used herein, “disease”, “disorder” and “condition” are used interchangeably, to indicate an abnormal state in a subject. In one embodiment, the disease is retinopathy, such as diabetic retinopathy. In another embodiment, the disease is glaucoma.

While various embodiments in the specification are presented using “comprising” language, under various circumstances, a related embodiment is also described using “consisting of” or “consisting essentially of” language. “Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

The terms “a” or “an” refers to one or more. For example, “an expression cassette” is understood to represent one or more such cassettes. As such, the terms “a” (or “an”), “one or more,” and “at least one” are used interchangeably herein.

As used herein, the term “about” means a variability of plus or minus 10% from the reference given, unless otherwise specified.

### EXAMPLES

The following examples disclose specific embodiments of recombinant NMDA antibodies and methods of use thereof. These examples should be construed to encompass any and all variations that become evident as a result of the teaching provided herein.

### Example 1: Reduction of Steady State Calcium Levels in A459 Lung Cancer MCF7 Breast Cancer Cell Lines Demonstrates the Anti-NMDAR Activity of the mAbs

The 5F5 and 2G6 mAbs antibodies were tested for their agonist or antagonist activity on A459 or MCF7 cell lines, which express functional NMDARs 50,000 cells per well were plated in black 96 well flat bottom plates (Thermo Fisher) and incubated overnight at 37° C. Cells then washed twice with Hanks balanced Solution (HBSS) without Ca⁺² or Mg⁺² (Thermo Fisher). Fluo-4 dye solution (ENZO lifesciences, FluoForte Calcium assay kit, Thermo Fisher) supplemented with 20 mM HEPES buffer was prepared according to the manufacturer's recommendations and 100 μl of the mixture was added to the cells. The cells were incubated at 37° C. for 45 min, then 15 min at room temp, and then washed twice with HBSS without Ca⁺². Ca⁺² fluorescence signal was measured at 490 excitation/525 emission using Cytation Microplate reader (BioTek Instruments). The plate then washed twice with 100 μl of HBSS without Ca⁺² or Mg⁺² and 100 μl of HBSS with Ca⁺² and Mg⁺² was added to the plate and incubated for 30 min at 37° C., at which time the Ca⁺² levels were again measured, as previously described. The mAbs (or MK-801 at 20 μM, Sigma Aldrich) were diluted to 5 μg/ml in HBSS with Ca⁺² and Mg⁺² and were added to the cells (triplicate samples) and incubated for 1 h at 37° C. followed; then Ca⁺² level was again measured. The plate was washed, and cells were incubated with a combination of two agonists (30 μM NMDA) and 100 μM Glycine (Sigma-Aldrich) at 37° C. and Ca⁺² levels were measured after 90, 120 and 150 sec.

### Example 2: 2G6, and 5F5 mAbs Bind Different Sites on the NMDAR

We assessed the binding epitopes of the 2G6 and 5F5 mAbs. We tested whether they bind overlapping epitopes on GluN1, using a competitive binding assay with HEK293T-ATD cells, which express the amino terminal domain of GluN1. Sharma, R., Al-Saleem, F. H., Panzer, J., Lee, J., Puligedda, R. D., Felicori, L. F., Kattala, C. D., Rattelle, A. J., Ippolito, G., Cox, R. H., Lynch, D. R. and Dessain, S. K. (2018), Monoclonal antibodies from a patient with anti-NMDA receptor encephalitis. Ann Clin Transl Neurol, 5:935-951, which is incorporated herein by reference.

HEK293T-ATD cells (10⁵ cells/well) were tested in a Whole Cell ELISA with luminescent detection method. We biotinylated the 5F5 and 2G6 or 5F5 using the EZ-Link™ Hydrazide-Biotin kit (Thermo Fisher), then generated dilution series of 2G6 or 5F5 (50 μg/mL to 0.2 μg/mL) and added to them the plate for 1 h at 37° C., followed by three washes with PBST. We then added the biotinylated 5F5 or 2G6 mAbs at 5 μg/mL, or PBS, and incubated for 1 h at 37° C. followed by three washes with PBST. The Pierce Streptavidin Poly-HRP substrate (Thermo Fisher) was added at 1:2000 dilution and incubated for 1 h at 37° C. This was followed by SuperSignal ELISA Femto Substrate, (1:1 ratio) (Thermo Fisher) and relative luminescence values were measured using the Synergy II plate reader. Duplicate binding curves were plotted and the linear portions were used for analysis using Excel.

We measured binding of one mAb, biotinylated, in the presence of increasing concentrations of the other. In this assay, each mAb competed with itself for binding, but neither mAb interfered with the binding of the other, even at fivefold excess, indicating that the mAbs bind to different sites on the GluN1 ATD.

### Example 3: 1D1 Binds an Epitope Different than 2G6 and 5F5

Experiments similar to those done in Example 2 were used to test whether the 1D1 binds a site on the NMDAR ATD that overlaps 2G6 and 5F5. In these experiments, 1D1 binding was tested in competition with mixed 2G6 and 5F5 mAbs. 1D1 binding is not inhibited by 2G6 and 5F5 binding. Conversely, 2G6 and 5F5 binding are not inhibited by 1D1.

### Example 4: The mAbs of the Present Invention are Tested for their Ability to Reduce Tinnitus in an Animal Model

The salicylate-treated rat model is an accepted model for evaluating therapeutic candidates for tinnitus. 225-gram Long Evans rats receive intraperitoneal injections of salicylate 350 mg/kg, daily for 4 days. These rats are administered vehicle or antibody by intratympanic injection. Intratympanic injections of mAb are tested at Time zero (before the first salicylate injection) and after the 4th injection. Groups of 8 rats are tested that include an intratympanic vehicle control only, an isotype control (non-NMDAR binding) mAb, and 1-3 of the mAbs of the present invention. A readout is taken of each rat at 24 or 48 hours after the last injection of salicylate. Rats are tested with the GPIAS assay (Gap-Prepulse Inhibition of the Acoustic Startle reflex), in which auditory response to a startle stimulus is measured to assess total hearing. Hearing is validated by providing a pre-startle stimulus and confirming reduced response. Tinnitus is assessed with a GAP detection test. The startle response is again tested following a run-in phase in which background noise is played in continuous fashion or with a temporal gap (GAP test). Animals without tinnitus have a reduced response to startle in the GAP group, compared to the continuous background (non-Gap group). In contrast, the steady state levels of tinnitus mask the GAP in background noise, such that both groups essentially have continuous noise and no difference in the magnitude of the startle response is seen. Animals that receive one, two, or three mAbs, either before or after the four salicylate injections, demonstrate a diminished startle in the GAP background noise group.

### Example 5: Production of Chimeric Recombinant Anti-NMDAR Antibodies

We used the variable domain sequences obtained from the hybridomas to produce purified chimeric versions of the antibodies to facilitate experimentation and confirm the activity of the obtained sequences. Antibodies were expressed in chimeric form. Antibody heavy chains were expressed as chimeras, containing the original human amino acid sequences and IgG1 Fc CH1 domain, in fusion with the murine IgG2a hinge, CH2 and CH3 domains. The original light chain amino acid sequences were not modified. We produced the 5F5, 2G6, and 1D1 anti-NMDAR antibodies and a control 9H2 antibody (SEQ ID Nos: 13 and 14), which is specific for poliovirus (REF https://pubmed.ncbi.nlm.nih.gov/31765719/) in this format. Antibodies were produced by transient transfection of paired heavy chain and light chain plasmids in Expi293 cells in ~500 ml culture with 98% viability and 3×10⁶ cells/ml cell density suitable for transfections. One hundred ml transfections in 500 ml shake flasks were set up with 50 μg of LC+50 μg HC plasmid DNA and cultured for 4 days when viability reached ~65%. Culture supernatant was collected by pelleting cells at 1000×g, 15 min, 4° C. and supernatant was stored at 4° C. until purification. Antibodies were purified by FPLC, using the AKTA Purifier 10 with Unicorn version 5.11 workstation and a Cytiva MabSelect SuRe 1 ml column following standard protocols. Antibodies were eluted over a 10 CV linear gradient of PBS 7.2 to Elution Buffer (0.1 M Citrate pH 3.06). 1 ml×10 fractions were collected into fraction tubes containing 50 μl to 200 μl of 1 M Tris pH 8.8 neutralization buffer. Antibody was confirmed by SDS-PAGE analysis, then concentrated and diluted four times, to a final volume of 4 ml with 10 mM Na Phosphate, 150 mM NaCl, pH 7.4.

### Example 6: NMDAR Antibody Binding in Photoreceptors

The NMDAR GluN1 antigen is not expressed in the normal mouse retina. The monoclonal antibodies 5F5, 2G6, and 1D1 bind to the GluN1 antigen on an epitope conserved among mouse, rat, and human. The 9H2 antibody recognizes a poliovirus antigen. Immunofluorescence staining of mouse retinas was performed. Recombinant chimeric antibodies were used for this experiment, as described in Example 5.

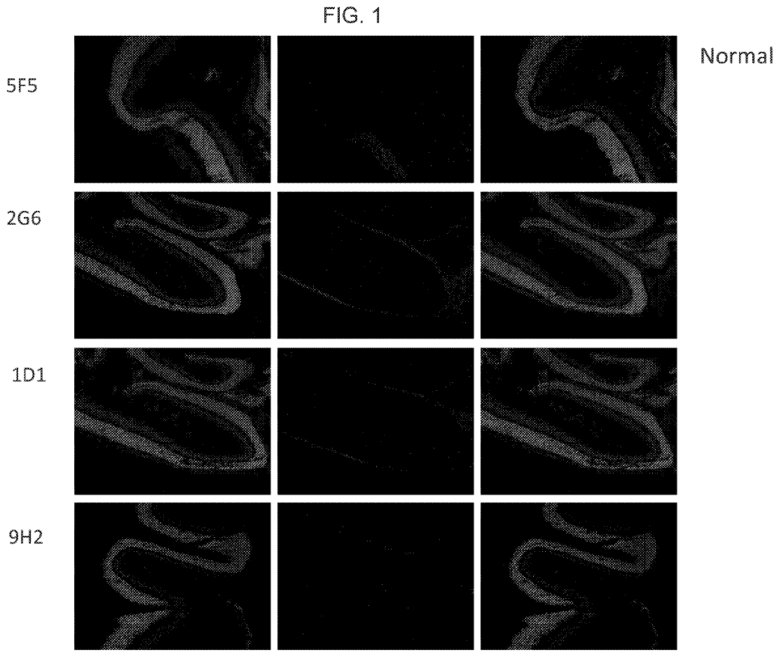

FIG. 1 shows antibody binding under normoxic conditions. No antibody binding is seen.

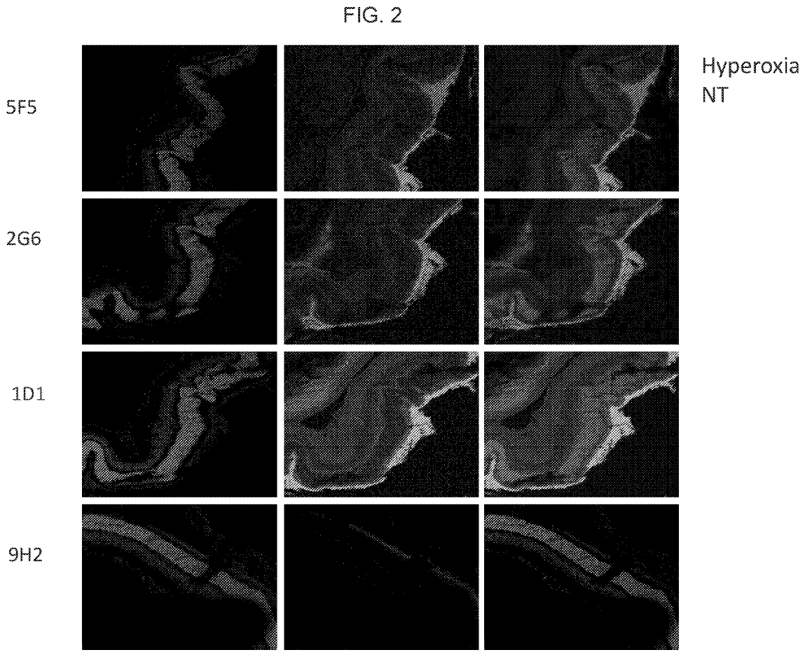

In contrast, the NMDAR GluN1 epitope recognized by the 5F5 antibody is expressed in the mouse retina exposed to hyperoxic conditions (the oxygen induced retinopathy model). Neonatal mice are exposed to hyperoxia (75% oxygen) from postnatal day 7 (P7) to P12, and then returned to room air. This treatment induces pathological vascular damage in the retina that causes hemorrhage. Immunofluorescence was performed with the 5F5 and 9H2 chimeric antibodies. The 5F5 antibody shows staining, whereas the control 9H2 antibody does not bind. (FIG. 2).

The NMDAR GluN1 epitopes recognized by the 2G6 and 1D1 antibodies are expressed in the mouse retina exposed to hyperoxic conditions (the oxygen induced retinopathy model). Neonatal mice were exposed to hyperoxia (75% oxygen) from postnatal day 7 (P7) to P12, and then returned to room air. Immunofluorescence was performed with the 2G6, 1D1, and 9H2 chimeric antibodies. The 5F5 antibody shows staining, whereas the control 9H2 antibody does not bind (FIG. 2).

### Example 7: The Anti NMDAR Antibody 5F5 Prevents Hyperoxia Induced Hemorrhage in the Mouse Oxygen Induced Retinopathy Model

Neonatal mice were exposed to hyperoxia (75% oxygen) from postnatal day 7 (P7) to P12, and then returned to room air. Upon return to room temperature, at P12, the eye on the right was injected with 5F5 antibody against NMDAR, while the left eye in the figure was injected with PBS. Consistent with vascular damage induced by the hyperoxic exposure, the untreated eye exhibits pathological retinal neovascularization and a leaky vessel, whereas the treated eye shows no bleeding. Whole eyes are shown in FIGS. 3A and 3B.

### Example 8: Retinitis Pigmentosa Model

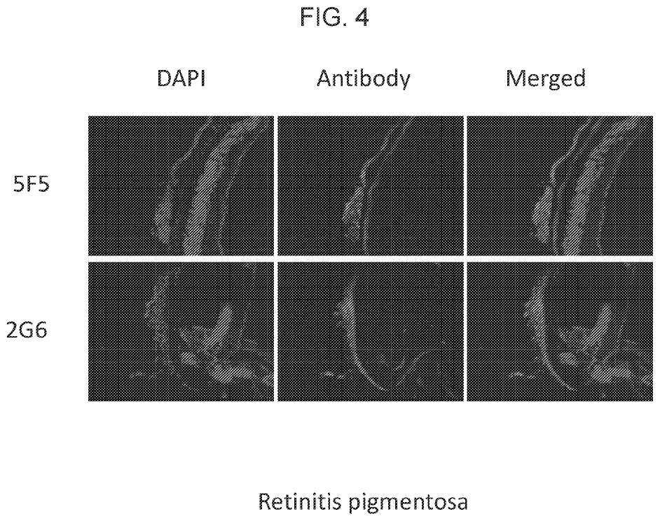

In the mouse model of retinitis pigmentosa, the NMDAR GluN1 epitopes recognized by the 5F5 and 2G6 antibodies are expressed in the mouse retina. FIG. 4.

### Example 9: Discussion

Immunofluorescence imaging demonstrated that the 5G5, 2G6, and 1D1 antibodies are immunoreactive with the outer segment of the photoreceptors as well as the choriocapillaris layers of the mouse retina. No retinal signal was seen with the isotype control 9H2 antibody (that is specific for a poliovirus antigen). This indicates that NMDAR is present within the retina and that it can be bound by our antibodies. (FIG. 2). Because the antibodies have potent NMDAR inhibitory effects, this provided a therapeutic hypothesis, namely, that NMDAR inhibition in the retina may protect retinal cells from injuries mediated by NMDAR activity. It is also notable that the disclosed antibodies bind an epitope that is conserved between murine and human NMDAR.

We next examined the potential effects of the NMDAR antibodies on pathological retinal neovascularization. We used the well characterized model of oxygen induced retinopathy (OIR) in mice. In this model, neonatal mice are exposed to hyperoxia (75% oxygen) from postnatal day 7 (P7) to P12, and then returned to room air. This approach, induces pathological neovascularization in the retina. In FIGS. 3A and 3B, a comparison is shown between two eyes of a mouse exposed to hyperoxia. Upon return to room temperature, at P12, the eye in FIG. 3B was injected with 5F5 antibody against NMDAR, while the eye in FIG. 3A was injected with PBS. Consistent with vascular damage induced by the hyperoxic exposure, the untreated eye exhibits pathological retinal neovascularization and a leaky vessel, whereas the treated eye shows no bleeding. This is a wholly unexpected finding.

Each patent, patent application, and publication, including websites cited throughout specification are incorporated herein by reference. Similarly, the SEQ ID NOS which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### Particular Embodiments

1. A method for treating retinopathy in a subject in need thereof, the method comprising administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

2. The method according to embodiment 1, said antibody or fragment comprising at least one of:
(a) a heavy chain sequence encoded by a nucleic acid sequence that is at least 85% identical to SEQ ID NOs. 2, 6, or 10; or
(b) a light chain sequence encoded by a nucleic acid sequence that is at least 85% identical to SEQ ID NOs: 4, 8, or 12; or
(c) a heavy chain sequence having an amino acid sequence that is at least 85% identical to SEQ ID NOs. 1, 5, or 9; or
(d) a light chain sequence having an amino acid sequence that is at least 85% identical to SEQ ID NOs: 3, 7, or 11, wherein the pharmaceutical composition is effective to suppress or reduce NMDA receptor mediated aberrant activity of the auditory nerve in the subject.

3 The method according to embodiment 1, wherein the pharmaceutical composition comprises
an antibody that comprises a heavy chain comprising SEQ ID NO: 1 and a light chain sequence comprising sequence SEQ ID NO: 3; or
an antibody that comprises a heavy chain sequence comprising SEQ ID NO: 5 and a light chain sequence comprising SEQ ID NO: 7; or
an antibody that comprises a heavy chain sequence comprising SEQ ID NO: 9 and a light chain sequence comprising SEQ ID NO: 11.

4. The method according to any one of embodiments 1 to 3, wherein the pharmaceutical composition comprises a 5F5, 1D1, or 2G6 antibody.

5. The method according to any one of embodiments 1 to 4, wherein the pharmaceutical composition comprises multiple antibodies.

6. The method according to embodiment 5, wherein the multiple antibodies bind to non-overlapping sites on the NMDAR.

7. The method according to any one of embodiments 1 to 6, wherein the epitope is located in the extracellular amino terminal domain (ATD) of Glutamate Ionotropic Receptor (GluN1) of the NMDAR.

8. The method according to any one of embodiments 1 to 7, wherein said epitope overlaps with epitopes recognized by autoimmune antibodies produced in a subject having Anti-N-methyl-D-aspartate Receptor Encephalitis (ANRE).

9 The method according to any one of embodiments 1 to 8, wherein said antibody is a chimeric antibody, a humanized antibody, a human antibody, a CDR-grafted antibody, a multispecific binding construct that can bind two or more targets, a dual specific antibody, a bi-specific antibody or a multi-specific antibody, or an affinity matured antibody, a single-domain antibody (sdAb), a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′) 2 construct, a monovalent or bivalent construct from which domains non-essential to monoclonal antibody function have been removed, a single-chain molecule containing one VL, one VH antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, or a monobody.

10. The method according to any one of embodiments 1 to 9, wherein the pharmaceutical composition comprises:
(a) antibodies 5F5 and 2G6 or epitope binding fragments thereof; or
(b) antibodies 5F5 and 1D1 or epitope binding fragments thereof; or
(c) antibodies 1D1 and 2G6 or epitope binding fragments thereof, or
(d) antibodies 5F5, 1D1 and 2G6 or epitope binding fragments thereof.

11. The method according to any one of embodiments 1 to 10, wherein the composition is administered to the eye.

12. The method according to embodiment 11, wherein the composition is administered by subretinal injection.

13. The method according to embodiment 11, wherein the composition is administered by intravitreal injection.

14. The method according to any one of embodiments 1 to 13, wherein the retinopathy is glaucoma or diabetic retinopathy.

15. The method according to any one of embodiments 1 to 13, wherein the retinopathy is retinitis pigmentosa.

16. The method according to any one of embodiments 1 to 13, wherein the retinopathy is related to aberrant vascularization.

17. A method for treating retinal bleeding in a subject in need thereof, the method comprising administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

18. The method according to any one of embodiments 1 to 17, wherein the pharmaceutical composition is administered with a secondary therapy.

19. The method according to embodiment 18, wherein the subject has retinopathy, and the secondary therapy is a VEGF antibody.

20. The method according to any one of embodiments 1 to 19, wherein the retinopathy is a vascular or proliferative retinopathy.

21. A method for treating tinnitus in a subject in need thereof, the method comprising administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

22. The method according to embodiment 21, said antibody or fragment comprising at least one of:
(a) a heavy chain sequence encoded by a nucleic acid sequence that is at least 85% identical to SEQ ID NOs. 2, 6, or 10; or
(b) a light chain sequence encoded by a nucleic acid sequence that is at least 85% identical to SEQ ID NOs: 4, 8, or 12; or
(c) a heavy chain sequence having an amino acid sequence that is at least 85% identical to SEQ ID NOs. 1, 5, or 9; or
(d) a light chain sequence having an amino acid sequence that is at least 85% identical to SEQ ID NOs: 3, 7, or 11,
wherein the pharmaceutical composition is effective to suppress or reduce NMDA receptor mediated aberrant activity of the auditory nerve in the subject.

23. The method according to embodiment 21, wherein the pharmaceutical composition comprises
an antibody that comprises a heavy chain comprising SEQ ID NO: 1 and a light chain sequence comprising sequence SEQ ID NO: 3; or
an antibody that comprises a heavy chain sequence comprising SEQ ID NO: 5 and a light chain sequence comprising SEQ ID NO: 7; or
an antibody that comprises a heavy chain sequence comprising SEQ ID NO: 9 and a light chain sequence comprising SEQ ID NO: 11.

24. The method according to any one of embodiments 21 to 23, wherein the pharmaceutical composition comprises a 5F5, 1D1, or 2G6 antibody.

25. The method according to any one of embodiments 21 to 24, wherein the pharmaceutical composition comprises multiple antibodies.

26. The method according to embodiment 25, wherein the multiple antibodies bind to non-overlapping sites on the NMDAR.

27. The method according to any one of embodiments 21 to 24 wherein the epitope is located in the extracellular amino terminal domain (ATD) of Glutamate Ionotropic Receptor (GluN1) of the NMDAR.

28. The method according to any one of embodiments 21 to 27, wherein said epitope overlaps with epitopes recognized by autoimmune antibodies produced in a subject having Anti-N-methyl-D-aspartate Receptor Encephalitis (ANRE).

29. The method according to any one of embodiments 21 to 28, wherein said antibody is a chimeric antibody, a humanized antibody, a human antibody, a CDR-grafted antibody, a multispecific binding construct that can bind two or more targets, a dual specific antibody, a bi-specific antibody or a multi-specific antibody, or an affinity matured antibody, a single-domain antibody (sdAb), a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, a monovalent or bivalent construct from which domains non-essential to monoclonal antibody function have been removed, a single-chain molecule containing one VL, one VH antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, or a monobody.

30. The method according to any one of embodiments 21 to 29, wherein the pharmaceutical composition comprises:
(a) antibodies 5F5 and 2G6 or epitope binding fragments thereof, or
(b) antibodies 5F5 and 1D1 or epitope binding fragments thereof; or
(c) antibodies 1D1 and 2G6 or epitope binding fragments thereof; or
(d) antibodies 5F5, 1D1 and 2G6 or epitope binding fragments thereof.

31. The method according to any one of embodiments 21 to 30, wherein the composition is administered to the inner ear.

32. The method according to embodiment 31, wherein the composition is administered by intratympanic injection.

33. The method according to embodiment 31, wherein the composition is administered locally via the round window membrane or oval window membrane into the inner ear.
1. Sharma R, Al-Saleem F H, Panzer J, Lee J, Puligedda R D, Felicori L F, et al. Monoclonal antibodies from a patient with anti-NMDA receptor encephalitis. Ann Clin Transl Neurol. 2018; 5 (8): 935-51. Epub 2018 Aug. 22. doi: 10.1002/acn3.592. PubMed PMID: 30128318; PubMed Central PMCID: PMCPMC6093837.
2. Sharma R, Al-Saleem F H, Puligedda R D, Rattelle A, Lynch D R, Dessain S K. Membrane-bound and soluble forms of an NMDA receptor extracellular domain retain epitopes targeted in auto-immune encephalitis. BMC biotechnology. 2018; 18 (1): 41. doi: 10.1186/s12896018-0450-1. PubMed PMID: 29945600; PubMed Central PMCID: PMCPMC6020338.
3. Hearn J I, Green T N, Hisey C L, Bender M, Josefsson E C, Knowlton N, et al. Deletion of Grin1 in mouse megakaryocytes reveals NMDA receptor role in platelet function and proplatelet formation. Blood. 2022; 139 (17): 2673-90. doi: 10.1182/blood.2021014000. PubMed PMID: 35245376.
4. Hughes E G, Peng X, Gleichman A J, Lai M, Zhou L, Tsou R, et al. Cellular and synaptic mechanisms of anti-NMDA receptor encephalitis. J Neurosci. 2010; 30 (17): 5866-75. Epub 2010 Apr. 30. doi: 10.1523/JNEUROSCI.0167-10.2010. PubMed PMID: 20427647; PubMed Central PMCID: PMC2868315.
5. Moscato E H, Peng X, Jain A, Parsons T D, Dalmau J, Balice-Gordon R J. Acute mechanisms underlying antibody effects in anti-N-methyl-D-aspartate receptor encephalitis. Ann Neurol. 2014; 76 (1): 108-19. Epub 2014 Jun. 12. doi: 10.1002/ana.24195. PubMed PMID: 24916964; PubMed Central PMCID: PMCPMC4296347.
6. Smith S B. Diabetic Retinopathy and the NMDA Receptor. Drug News Perspect. 2002; 15 (4): 226-32. Epub 2003 Apr. 5. doi: 10.1358/dnp.2002.15.4.840055. PubMed PMID: 12677206; PubMed Central PMCID: PMCPMC3773688.
7. Cheung W, Guo L, Cordeiro M F. Neuroprotection in glaucoma: drug-based approaches. Optom Vis Sci. 2008; 85 (6): 406-16. Epub 2008 Jun. 4. doi: 10.1097/OPX.0b013e31817841e5. PubMed PMID: 18521010; PubMed Central PMCID: PMCPMC2597725.
8. Ferreira I L, Duarte C B, Carvalho A P. Ca2+ influx through glutamate receptor-associated channels in retina cells correlates with neuronal cell death. Eur J Pharmacol. 1996; 302 (1-3): 15362. Epub 1996 Apr. 29. doi: 10.1016/0014-2999 (96) 00044-1. PubMed PMID: 8791003.
9. Niwa M, Aoki H, Hirata A, Tomita H, Green P G, Hara A. Retinal Cell Degeneration in Animal Models. Int J Mol Sci. 2016; 17 (1). Epub 2016 Jan. 20. doi: 10.3390/ijms17010110. PubMed PMID: 26784179; PubMed Central PMCID: PMCPMC4730351.
10. Ganapathy P S, White R E, Ha Y, Bozard B R, McNeil P L, Caldwell R W, et al. The role of N-methyl-D-aspartate receptor activation in homocysteine-induced death of retinal ganglion cells. Invest Ophthalmol Vis Sci. 2011; 52 (8): 5515-24. Epub 2011 Mar. 26. doi: 10.1167/iovs. 106870. PubMed PMID: 21436276; PubMed Central PMCID: PMCPMC3176067.
11. Gabelt B T, Rasmussen C A, Tektas O Y, Kim C B, Peterson J C, Nork T M, et al. Structure/function studies and the effects of memantine in monkeys with experimental glaucoma. Invest Ophthalmol Vis Sci. 2012; 53 (4): 2368-76. Epub 2012 Mar. 20. doi: 10.1167/iovs.11-8475. PubMed PMID: 22427549; PubMed Central PMCID: PMCPMC3833458.
12. Santiago A R, Hughes J M, Kamphuis W, Schlingemann R O, Ambrosio A F. Diabetes changes ionotropic glutamate receptor subunit expression level in the human retina. Brain Res. 2008; 1198:153-9. Epub 2008 Feb. 9. doi: 10.1016/j.brainres.2007.12.030. PubMed PMID: 18258217.
13. Kusari J, Zhou S, Padillo E, Clarke K G, Gil D W. Effect of memantine on neuroretinal function and retinal vascular changes of streptozotocin-induced diabetic rats. Invest Ophthalmol Vis Sci. 2007; 48 (11): 5152-9. Epub 2007 Oct. 27. doi: 10.1167/iovs.07-0427. PubMed PMID: 17962468.
14. Dalmau J, Gleichman A J, Hughes E G, Rossi J E, Peng X, Lai M, et al. Anti-NMDAreceptor encephalitis: case series and analysis of the effects of antibodies. The Lancet Neurology. 2008; 7 (12): 1091. Epub 2008 Oct. 15. doi: S1474-4422 (08) 70224-2 [pii] 10.1016/S1474-4422 (08) 70224-2. PubMed PMID: 18851928.
15. Adamis A P, Brittain C J, Dandekar A, Hopkins J J. Building on the success of antivascular endothelial growth factor therapy: a vision for the next decade. Eye (Lond). 2020; 34 (11): 1966-72. Epub 20200615. doi: 10.1038/s41433-020-0895-z. PubMed PMID: 32541890; PubMed Central PMCID: PMCPMC7784857.
16. Meissirel C, Ruiz de Almodovar C, Knevels E, Coulon C, Chounlamountri N, Segura I, et al. VEGF modulates NMDA receptors activity in cerebellar granule cells through Src-family kinases before synapse formation. Proc Natl Acad Sci USA. 2011; 108 (33): 13782-7. Epub 2011 Aug. 2. doi: 10.1073/pnas. 1100341108. PubMed PMID: 21804034; PubMed Central PMCID: PMCPMC3158143.
17. De Rossi P, Harde E, Dupuis J P, Martin L, Chounlamountri N, Bardin M, et al. A critical role for VEGF and VEGFR2 in NMDA receptor synaptic function and fear-related behavior. Mol Psychiatry. 2016; 21 (12): 1768-80. Epub 2016 Jan. 6. doi: 10.1038/mp.2015.195. PubMed PMID: 26728568; PubMed Central PMCID: PMCPMC5116482.

1. A method for treating retinopathy in a subject in need thereof, the method comprising administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

2. The method according to claim 1, said antibody or fragment comprising at least one of:
(a) a heavy chain sequence encoded by a nucleic acid sequence that is at least 85% identical to SEQ ID NO: 2, 6, or 10; or
(b) a light chain sequence encoded by a nucleic acid sequence that is at least 85% identical to SEQ ID NO: 4, 8, or 12; or
(c) a heavy chain sequence having an amino acid sequence that is at least 85% identical to SEQ ID NO; 1, 5, or 9; or
(d) a light chain sequence having an amino acid sequence that is at least 85% identical to SEQ ID NO: 3, 7, or 11,
wherein the pharmaceutical composition is effective to suppress or reduce NMDA receptor mediated aberrant activity of the auditory nerve in the subject.

3. The method according to claim 1, wherein the pharmaceutical composition comprises
an antibody that comprises a heavy chain comprising SEQ ID NO: 1 and a light chain sequence comprising sequence SEQ ID NO: 3; or
an antibody that comprises a heavy chain sequence comprising SEQ ID NO: 5 and a light chain sequence comprising SEQ ID NO: 7; or
an antibody that comprises a heavy chain sequence comprising SEQ ID NO: 9 and a light chain sequence comprising SEQ ID NO: 11.

4. The method according to claim 1, wherein the pharmaceutical composition comprises a 5F5, 1D1, or 2G6 antibody.

5. The method according to claim 1, wherein the pharmaceutical composition comprises multiple antibodies.

6. The method according to claim 5, wherein the multiple antibodies bind to non-overlapping sites on the NMDAR.

7-8. (canceled)

9. The method according to claim 1, wherein said antibody is a chimeric antibody, a humanized antibody, a human antibody, a CDR-grafted antibody, a multispecific binding construct that can bind two or more targets, a dual specific antibody, a bi-specific antibody or a multi-specific antibody, or an affinity matured antibody, a single-domain antibody (sdAb), a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, a monovalent or bivalent construct from which domains non-essential to monoclonal antibody function have been removed, a single-chain molecule containing one VL, one VH antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, or a monobody.

10. The method according to claim 1, wherein the pharmaceutical composition comprises:
(a) antibodies 5F5 and 2G6 or epitope binding fragments thereof; or
(b) antibodies 5F5 and 1D1 or epitope binding fragments thereof, or
(c) antibodies 1D1 and 2G6 or epitope binding fragments thereof; or
(d) antibodies 5F5, 1D1 and 2G6 or epitope binding fragments thereof.

11. The method according to claim 1, wherein the composition is administered to the eye.

12-16. (canceled)

17. A method for treating retinal bleeding in a subject in need thereof, the method comprising administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

18. The method according to claim 1, wherein the pharmaceutical composition is administered with a secondary therapy.

19. The method according to claim 18, wherein the subject has retinopathy, and the secondary therapy is a VEGF antibody.

20. The method according to claim 1, wherein the retinopathy is a vascular or proliferative retinopathy.

21. A method for treating tinnitus in a subject in need thereof, the method comprising administering an effective amount of a pharmaceutical composition comprising a recombinant, synthetic or monoclonal human antibody or an epitope binding fragment thereof that specifically binds to an N-methyl-D-aspartate Receptor (NMDAR) epitope.

22. The method according to claim 21, said antibody or fragment comprising at least one of:
(a) a heavy chain sequence encoded by a nucleic acid sequence that is at least 85% identical to SEQ ID NO: 2, 6, or 10; or
(b) a light chain sequence encoded by a nucleic acid sequence that is at least 85% identical to SEQ ID NO: 4, 8, or 12; or
(c) a heavy chain sequence having an amino acid sequence that is at least 85% identical to SEQ ID NO: 1, 5, or 9; or
(d) a light chain sequence having an amino acid sequence that is at least 85% identical to SEQ ID NO: 3, 7, or 11,
wherein the pharmaceutical composition is effective to suppress or reduce NMDA receptor mediated aberrant activity of the auditory nerve in the subject.

23. The method according to claim 21, wherein the pharmaceutical composition comprises
an antibody that comprises a heavy chain comprising SEQ ID NO: 1 and a light chain sequence comprising sequence SEQ ID NO: 3; or
an antibody that comprises a heavy chain sequence comprising SEQ ID NO: 5 and a light chain sequence comprising SEQ ID NO: 7; or
an antibody that comprises a heavy chain sequence comprising SEQ ID NO: 9 and a light chain sequence comprising SEQ ID NO: 11.

24. The method according to claim 21, wherein the pharmaceutical composition comprises a 5F5, 1D1, or 2G6 antibody.

25-30. (canceled)

31. The method according to claim 21, wherein the composition is administered to the inner ear.

32. The method according to claim 31, wherein the composition is administered by intratympanic injection.

33. The method according to claim 31, wherein the composition is administered locally via the round window membrane or oval window membrane into the inner ear.


SEQUENCE LISTING (12 sequences)

SEQ ID NO: 1 - protein, 128 aa, Mus musculus
QVQLVESGGGVVRPGGSLRLSCAASGFTFSTYSLHWVRQAPGKGLEWVGVILYDGSKKYYADSVRGRFTISRDNSKSTLNLDMSSLRPDDTAVYYCARDPIAVAPRPSGMVPQGFDYWGQGTLVTVSS

SEQ ID NO: 2 - DNA, 1434 bp, Mus musculus
  source 1..1434: mol_type = unassigned DNA
atgaaacatctctggttctttctccttttggtcgcagctccccgttgggttctctcccaggtgcagctggtggagtctggaggaggcgtggtccggcctggggggtccctgagactctcttgtgcagcctctggattcaccttcagtacctatagtcttcactgggtccgccaggctccaggcaaggggctggagtgggttggagttattttatatgatggcagcaaaaaatattatgcagactccgtgaggggccgattcaccatctccagagacaattccaagagcacgctaaatctggatatgagcagcctgagacctgacgacacggctgtgtattactgtgcgagagacccaatagcagtggctcccaggcccagcggcatggtcccccagggatttgactattggggccagggaaccctggtcaccgtctcctcagcctccaccaagggacctagcgtgttcccactggctccctctagtaaaagtacatcagggggaactgcagccctgggctgtctggtgaaggattactttccagagcccgtcacagtgagctggaactccggggccctgacaagcggagtccatacttttccagctgtgctgcagtcaagcggactgtactccctgtcctctgtggtcactgtgcctagttcaagcctgggaacacagacttatatttgcaacgtgaatcacaagccatccaataccaaagtggacaagaaagtggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 3 - protein, 110 aa, Homo sapiens
QSVVTQPPSASGTPGQRVTISCSGSSSNIGRNFVFWYRQLPGTAPKVLIYKNIQRPSGVPDRISGSRSGSSASLAISGLRSEDEADYYCASWDDSLSGVVFGGGTKLTVL

SEQ ID NO: 4 - DNA, 705 bp, Homo sapiens
  source 1..705: mol_type = unassigned DNA
atggcctgggccacgctccttcttcctcttttgaatctctacactggttctgtcgcccagtctgtcgtgacgcagccgccctcagcgtctgggacccccgggcagagggtcaccatctcctgttctggaagcagctccaacatcggaagaaattttgtattctggtatcggcagctcccaggaacggcccccaaagtcctcatctataagaatattcagcggccctcaggggtccctgaccgaatctctggctccaggtctggctcctcagcctccctggccatcagtggactccggtccgaggatgaggctgattattactgtgcatcatgggatgacagcctgagtggtgtggtgttcggcggggggaccaagctgaccgtcctaggccaaccaaaagcaaatccaacggtgacgctgtttccgccatcttcagaagaattgcaggctaataaagctactctggtatgcctcatttccgatttctatccaggggccgtcaccgtagcttggaaagccgacgggtcaccagtgaaagcaggcgtcgaaacaacgaagcctagcaagcaatccaataataaatatgctgcttcttcatatttgtctcttaccccagaacaatggaaaagccatcgctcttattcatgtcaagtgactcacgagggctctactgtagaaaagactgtagctccgacggagtgctcc

SEQ ID NO: 5 - protein, 120 aa, Mus musculus
LVQLVESGGGVVQSGRSLRLSCAASGFAFHTFTIHWVRQAPGKGLDWVTAIYFDGTKKYYADSVKGRFTVSRDNSKNTVYLQMNGLRGEDTAVYYCARARYSYGLSFDYWGQGTPVTVSS

SEQ ID NO: 6 - DNA, 1419 bp, Mus musculus
  source 1..1419: mol_type = unassigned DNA
atgaaacatctctggttctttctccttttggtcgcagctccccgttgggttctctccgtccagtgtcaggtgcagctggtggagtccgggggaggcgtggtccagtctgggaggtccctgagactctcctgtgcagcctctggattcgccttccatacctttactatacactgggtccgccaggctccaggcaaggggctggactgggtaacagctatatattttgatggaaccaaaaaatactacgcagactccgtgaagggccgattcaccgtctccagagacaactccaagaacacggtatatctgcaaatgaacggcctgagaggtgaggacacggctgtctattactgtgcgagagcccgatacagctatggcctttcctttgactactggggccagggaaccccggtcaccgtctcctctgcctccaccaagggacctagcgtgttcccactggctccctctagtaaaagtacatcagggggaactgcagccctgggctgtctggtgaaggattactttccagagcccgtcacagtgagctggaactccggggccctgacaagcggagtccatacttttccagctgtgctgcagtcaagcggactgtactccctgtcctctgtggtcactgtgcctagttcaagcctgggaacacagacttatatttgcaacgtgaatcacaagccatccaataccaaagtggacaagaaagtggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 7 - protein, 110 aa, Homo sapiens
QSVVTQPPSVSAAPGQKVTISCSASSSYLGSNYVSWYQQLPGTAPKLLIYDNNKRSSGISDRFSGSKSGTSATLDITGLQTGDEADYYCGAWDSSLSVVVFGGGTKLTVL

SEQ ID NO: 8 - DNA, 705 bp, Homo sapiens
  source 1..705: mol_type = unassigned DNA
atggcctgggccacgctccttcttcctcttttgaatctctacactggttctgtcgcccagtctgtcgtgacgcagccgccctcagtgtctgcggccccaggacagaaggtcaccatctcctgctctgcaagcagctcctaccttgggagtaattatgtatcttggtaccagcaactcccaggaacagcccccaaactcctcatttatgacaataataagcgatcctcagggatttctgaccgattctctggctccaagtctggcacgtcagccaccctggacatcaccggcctgcagactggggacgaggccgactattactgcggagcatgggacagcagcctgagtgtcgtggttttcggcggagggaccaagctgaccgtcctaggccaaccaaaagcaaatccaacggtgacgctgtttccgccatcttcagaagaattgcaggctaataaagctactctggtatgcctcatttccgatttctatccaggggccgtcaccgtagcttggaaagccgacgggtcaccagtgaaagcaggcgtcgaaacaacgaagcctagcaagcaatccaataataaatatgctgcttcttcatatttgtctcttaccccagaacaatggaaaagccatcgctcttattcatgtcaagtgactcacgagggctctactgtagaaaagactgtagctccgacggagtgctcc

SEQ ID NO: 9 - protein, 130 aa, Mus musculus
QVQLQESGGGVVQPGRSLRLSCAASGFSFNAFAMHWVRQAPGKGLEWVARISHYGSDDYYADSVKGRFTISRDNSQNTLFLQMNSLKAEDTGVYYCWRGFTLVRGVISRNPINRFSGMDVWGQGTTVTVS

SEQ ID NO: 10 - DNA, 1443 bp, Mus musculus
  source 1..1443: mol_type = unassigned DNA
atgaaacatctctggttctttctccttttggtcgcagctccccgttgggttctctcccaggttcagttgcaagagtcaggcggtggagtcgtgcagccgggacgcagtcttaggcttagctgcgccgcgtctgggtttagctttaacgcctttgccatgcattgggttagacaggcccctggcaagggactcgagtgggtcgccagaataagccattatggtagtgacgactactatgctgattctgttaagggtcgatttaccattagtcgagataactcacagaacacgttgttcttgcagatgaactctcttaaagctgaggatacaggagtatactattgttggcgcggcttcactctggtgcgcggcgtaatttccagaaatccaattaatagattcagtgggatggacgtctggggacagggcaccactgtaacagttagctccgcctccaccaagggacctagcgtgttcccactggctccctctagtaaaagtacatcagggggaactgcagccctgggctgtctggtgaaggattactttccagagcccgtcacagtgagctggaactccggggccctgacaagcggagtccatacttttccagctgtgctgcagtcaagcggactgtactccctgtcctctgtggtcactgtgcctagttcaagcctgggaacacagacttatatttgcaacgtgaatcacaagccatccaataccaaagtggacaagaaagtggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 11 - protein, 108 aa, Homo sapiens
SYELMQPPSVSVSPGQTARITCSGDALPKKYAYWYQQKSGQAPILVIYEDNKRPSGIPERFSGSSSGTMATLTISGAQVEDEADYFCYSTDSSGNHGVFGGGTKLTVL

SEQ ID NO: 12 - DNA, 699 bp, Homo sapiens
  source 1..699: mol_type = unassigned DNA
atggcctgggccacgctccttcttcctcttttgaatctctacactggttctgtcgcctcatatgaactgatgcaacccccatcagtctctgtaagtccgggacaaacagcacgaataacctgttcaggggacgctcttccaaaaaaatacgcctactggtatcagcaaaagagcggacaggcccctatcctcgttatatacgaagacaataaacgcccatctggtatacccgaacgcttttcaggtagtagctccgggacgatggcgaccctgacaatatcaggtgctcaagtcgaagatgaggcagactacttttgctattctaccgattcatctggaaaccatggagtctttggcggcggtacaaaactcacagtgcttggccaaccaaaagcaaatccaacggtgacgctgtttccgccatcttcagaagaattgcaggctaataaagctactctggtatgcctcatttccgatttctatccaggggccgtcaccgtagcttggaaagccgacgggtcaccagtgaaagcaggcgtcgaaacaacgaagcctagcaagcaatccaataataaatatgctgcttcttcatatttgtctcttaccccagaacaatggaaaagccatcgctcttattcatgtcaagtgactcacgagggctctactgtagaaaagactgtagctccgacggagtgctcc